SEQUENCE LISTING (6 sequences)

SEQ ID NO: 1 - DNA, 19 bp, Artificial
  Synthetic DNA
gaaaacgggtatgtggaga

SEQ ID NO: 2 - DNA, 19 bp, Artificial
  Synthetic DNA
tgaaacagacggatggtga

SEQ ID NO: 3 - DNA, 19 bp, Artificial
  Synthetic DNA
tggtggacgctcttcagtt

SEQ ID NO: 4 - DNA, 19 bp, Artificial
  Synthetic DNA
catctccagcctcctcaga

SEQ ID NO: 5 - DNA, 17 bp, Artificial
  Synthetic DNA
ctacaatggacagcaat

SEQ ID NO: 6 - DNA, 23 bp, Artificial
  Synthetic DNA
gaaactctctctctttctgttgc

(19) United States
(12) Patent Application Publication
(10) Pub. No.: US 2006/0257935 A1
(43) Pub. Date: Nov. 16, 2006

(54) Hematopoietic factor production promoter

(75) Inventors: Sen Takeshita, Kawasaki-shi (JP); Yuzuru Eto, Kawasaki-shi (JP); Yoshiyuki Takahara, Kawasaki-shi (JP); Kiyonobu Kamiya, Kawasaki-shi (JP); Kazumi Tashiro, Kawasaki-shi (JP); Maki Sugiyama, Kawasaki-shi (JP); Hiroshi Yamamoto, Kawasaki-shi (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Ajinomoto Co., Inc., Tokyo (JP)

(21) Appl. No.: 11/410,948

(22) Filed: Apr. 26, 2006

Related U.S. Application Data
(60) Provisional application No. 60/678,178, filed on May 6, 2005.

(30) Foreign Application Priority Data
Apr. 26, 2005 (JP) 2005-128204

(51) Int. Cl. G01N 33/53

(52) U.S. Cl. 435/7.1

(57) ABSTRACT

The present invention provides methods of screening for enhancers and/or inhibitors of a hematopoietic factor production promoter comprising arginine as an active ingredient. Arginine is an excellent hematopoietic factor production promoter, because it has high safety, can be orally administered, and is widely used.

### CROSS REFERENCES TO RELATED APPLICATIONS

This application claims priority to U.S. Provisional Patent Application No. 60/678,178, filed on May 6, 2005, and Japanese Patent Application No. 2005-128204, filed on Apr. 26, 2905, both of which are incorporated herein by reference in their entireties.

### BACKGROUND OF THE INVENTION

1. Field of the Invention

The present invention relates to a hematopoietic factor production promoter and a method of screening an enhancer or inhibitor for the hematopoietic factor production promoter.

2. Discussion of the Background

Erythropoiesis is an essential process required to maintain homeostasis of the number of red blood cells. Red blood cells have an average life span of about 120 days in humans, and worn out red blood cells are continuously removed from the circulation system. Therefore, about 100 billion red blood cells are newly produced daily in adults. There have been many investigations of the generation of red blood cells, which are described in a number of books. As a typical example, a summary is excerpted from “Hematology, Chugai Igakusha”, which will be shown below.

There are multipotent stem cells capable of differentiating into many different types of blood cell systems in the bone marrow, and a portion of the multipotent stem cells differentiate into erythroid progenitors determined to differentiate into the erythroid system. The most primitive identifiable erythroid progenitors are the burst forming unit-erythroid (BFU-E) and the colony forming unit-erythroid (CFU-E), which is a more differentiated cell. Beyond the CFU-E stage, the cells differentiate into proerythroblasts, basophilic erythroblasts, polychromatic erythroblasts, and orthochromatic erythroblasts while dividing, and reticulocytes differentiate into mature red blood cells by enucleation. At the BFU-E and CFU-E stages, the direction of differentiation into the erythroid system is completely determined, therefore the cells never differentiate into a blood cell system other than the erythroid system. Thus, as the number of BFU-E and CFU-E increases, erythropoiesis is promoted.

As for erythropoiesis, homeostasis is principally regulated by erythropoietin (EPO), which is a hematopoietic factor. EPO is mainly produced in the kidney and circulates in the blood and acts on CFU-E in the bone marrow and stimulates proliferation and differentiation of CFU-E, thereby promoting erythropoiesis. When EPO is not produced at a normal level and running short, the amount of CFU-E decreases and erythropoiesis is reduced thereby causing anemia. Anemia is the pathological consequence of insufficient hemoglobin levels to meet the oxygen transport requirements of the body and causes clinical symptoms such as less incentive to work, fatigability, shortness of breath, lightheadedness and palpitation, therefore there is a demand for improving such symptoms. It is known that various diseases cause anemia due to insufficient EPO, and the most classical example is a kidney disease. In patients with chronic renal failure, EPO production is reduced due to renal damage, whereby the patients exhibit anemia. Many of the patients with chronic renal failure are dialysis patients who require frequent dialysis for renal function replacement, and 90% of the dialysis patients have anemia. At present, as a method of treating anemia, administration of recombinant human EPO (rHuEPO) is widely used, and 90% of the dialysis patients are administered with rHuEPO. In many of the patients, an effect on improving anemia has been confirmed.

The effect of rHuEPO on improving anemia is high, however, several problems have arisen accompanying the expansion of the clinical use thereof. One is the cost for long-term treatment thereof. The typical dose of rHuEPO is 9000 IU/week at most, which costs about 12,000 yen on an NHI drug price basis. However, unless the primary disease causing anemia is treated, it is administered over a long period of time. Therefore, the burden on the patients and health care system is great in terms of the cost. Further, because EPO has to be intravenously administered, the patients need to go to the hospital every time they receive the administration, etc., which is also a cause of why the burden on patients is great. Further, it is known that there are about 10 to 20% of patients whose EPO level in the blood is increased by the administration of rHuEPO, but whose reactivity against EPO is low, therefore who need a high dose of rHuEPO for alleviating anemia. or whose anemia is hardly improved at all (these patients are called EPO-unresponsive patients). It is considered that for alleviating anemia in the EPO-unresponsive patients, a bone marrow erythroid progenitor cell(s) differentiation inducer, which increases CFU-E based on a mechanism of action which is different from that of EPO, would be effective. Further, by the concomitant use of the bone marrow erythroid progenitor cell(s) differentiation inducer, an effect of reducing the required dose of rHuEPO is also expected.

It is known that proliferation and differentiation of erythroid progenitors are induced by a plurality of hematopoietic factors other than EPO. It is known that activin and IGF-1, each of which is a hematopoietic factor, act on CFU-E synergistically with EPO and induce proliferation and differentiation thereof (Blood, 1992, Nov. 15; 80(10): 2503-12). It is also known that SCF, which is a hematopoietic factor, acts on BFU-E, which is an erythroid progenitor less differentiated than CFU-E and induces proliferation and differentiation thereof (Blood, 1991, Oct. 15; 78(8): 1975-80).

These hematopoietic factors, activin, IGF-1 and SCF act on a receptor different from that of EPO and increase CFU-E, therefore it is expected that anemia is improved by administration thereof to EPO-unresponsive patients. However, since administration of a recombinant protein of a hematopoietic factor is parenteral administration (like EPO), the burden of patients is great. For example, every time a parenteral dose administration is need, the patient would need to go to the hospital, often the trip itself further complicates the manifestation of the symptoms that the treatment attempts to alleviate. Further, it is known that when a plurality of hematopoietic factors act on concomitantly, a synergistic action thereof are exhibited in general, however, the concomitant use of a plurality of hematopoietic factors makes the burden of patients great in terms of cost.

Accordingly, it is expected that if a medicament that has an action of promoting the production of activin, IGF-1 and SCF simultaneously is discovered, the medicament will become a therapeutic agent for anemia that solves the current clinical problems.

In Int. J. Toxicol., 23: 101-105 (2004), it is described that the amount of hemoglobin and red blood cells was increased by repeatedly administering arginine to normal rats. In Igakuno Ayumi, 211, No. 8 (2004), it is described that the condition of anemia was improved (the number of red blood cells came close to a normal level) by administering arginine to patients with renal anemia. However, there is no description that arginine is capable of promoting the production of hematopoietic factors by directly acting on the bone marrow.

In J. Nutr., vol. 129, pp. 1298-1306 (1999), it is reported that by adding arginine, proline, threonine, and triptophan to a medium for porcine primary hepatocyte culture at a high concentration, gene expression of IGF-1 increases. This study was performed by limiting the actions of amino acids on hepatocytes, and there is no description of an action on cells other than hepatocytes or an action on erythropoiesis.

In Bone, vol. 23, pp. 103-109 (1998), it is reported that when arginine is added to MC3T3-E1 cells, which is an osteoblast-like cell line, the secretion amount of IGF-1 increases, and when arginine is added at a higher concentration, the gene expression increases.

It is also described that because osteoblasts are involved in the formation of bone, there may be a possibility that arginine promotes the formation of bone by enhancing IGF-1 production. That is, this study was performed by paying attention to the action on bone metabolism regulation, and there is no description of an action on erythropoiesis in the document.

### SUMMARY OF THE INVENTION

Accordingly, it is one object of the present invention to provide a hematopoietic factor production promoter.

It is another object of the present invention to provide a method of screening an enhancer or inhibitor for the hematopoietic factor production promoter.

More specifically, in a method of measuring CFU-E, the in vitro colony assay method using a methylcellulose semi-solid medium is generally used, and it is the most suitable experimental system for discovering an action of increasing CFU-E. The present inventors employed this method and measured the number of CFU-E colonies when 600 μM arginine was added to isolated mouse bone marrow cells, and as a result, they found that the number of CFU-E significantly increases compared with the case of an average concentration of arginine in the blood, 170 μM. In the case of oral administration to rats, the blood arginine concentration reaches about 600 μM by a single-dose administration of arginine at 1.2 g/kg. Therefore, it is expected that the number of CFU-E increases in a dose-dependent manner by such an oral administration. Also in humans, it is expected that the number of CFU-E increases as the concentration of arginine in the blood increases by the oral administration.

Further, in order to verify the mechanism of CFU-E-colony stimulating activity arginine, bone marrow cells were isolated from a renal failure model rat, and a gene expression analysis of hematopoietic factors after the addition of arginine was performed. As a result, it was determined that the gene expression of any of activin, SCF and IGF-1 is promoted.

Further, in order to verify whether the mechanism of CFU-E-colony stimulating activity of arginine resides in the promotion of production of activin, SCF or IGF-1, a mouse colony assay was performed. As a result, by the respective inhibitors for activin, SCF and IGF-1, the CFU-E-colony stimulating activity of arginine was inhibited. Accordingly, it determined that arginine can increase the number of CFU-E by promoting the production of activin, SCF and IGF-1.

From the above-mentioned results, the present inventors newly discovered that arginine serves to increase CFU-E whose mechanism of action resides in having an activity of promoting the production of activin, SCF and IGF-1. In other words, it becomes a differentiation promoter for erythroid progenitors, which is excellent in safety and can be orally administered, thus the present invention has been completed.

Accordingly, the present invention provides the following:

(1) A hematopoietic factor production, comprising arginine or a physiologically acceptable salt thereof as an active ingredient.

(2) The hematopoietic factor production promoter according to (1), wherein the hematopoietic factor is at least one selected from the group consisting of activin, SCF and IGF-1.

(3) The hematopoietic factor production promoter according to (1), which promotes production of hematopoietic factor in the bone marrow.

(4) A therapeutic agent for promoting production of hematopoietic factor in the bone marrow, comprising the hematopoietic factor production promoter according to (1).

(5) The therapeutic agent according to (4), which is for oral administration.

(6) The therapeutic agent according to (4), which is for parenteral administration.

(7) A method of screening for an enhancer for a hematopoietic factor production promoter which comprises arginine or a physiologically acceptable salt thereof as an active ingredient, wherein said method comprises:

(a-1) measuring hematopoietic factor production-promoting activities by adding the hematopoietic factor production promoter and a test substance to a hematopoietic factor production-promoting activity measurement system which is for measuring the hematopoietic factor production-promoting activity;

(a-2) measuring hematopoietic factor production-promoting activities by adding the hematopoietic factor production promoter to a hematopoietic factor production-promoting activity measurement system which is for measuring the hematopoietic factor production-promoting activity;

(b) comparing hematopoietic factor production-promoting activities of (a-1) to hematopoietic factor production-promoting activities of (a-2); and

(c) classifying the test substance as an enhancer for a hematopoietic factor production promoter when the hematopoietic factor production-promoting activity of (a-1) is greater than the hematopoietic factor production-promoting activity of (a-2).

(8) The method according to (7), wherein the hematopoietic factor production promoter and the test substance are added simultaneously.

(9) The method according to (7), wherein the hematopoietic factor production promoter and the test substance are added sequentially.

(10) The method according to (7), wherein said hematopoietic factor production-promoting activity measurement system is quantitative PCR.

(11) A method of enhancing production of hematopoietic factor in the bone marrow, comprising administering to a subject in need thereof an effective amount of a composition comprising a hematopoietic factor production promoter comprising arginine or a physiologically acceptable salt thereof and an enhancer for the hematopoietic factor production promoter identified by the method according to (7).

(12) The method according to (11), wherein said administering is orally.

(13) The method according to (11), wherein said administering is parenterally.

(14) The method according to (11), wherein arginine or a physiologically acceptable salt thereof is administered at a concentration of 0.1 to 12 g per day.

(15) The method according to (11), wherein arginine or a physiologically acceptable salt thereof is administered at a concentration of 0.5 to 6 g per day.

(16) The method according to (11), wherein the hematopoietic factor is at least one selected from the group consisting of activin, SCF and IGF-1.

(17) A method of treating a disease caused by a decrease in erythropoiesis, comprising administering to a subject in need thereof an effective amount of a composition comprising a hematopoietic factor production promoter comprising arginine or a physiologically acceptable salt thereof and an enhancer for the hematopoietic factor production promoter identified by the method according to (7).

(18) The method according to (17), wherein said administering is orally.

(19) The method according to (17), wherein said administering is parenterally.

(20) The method according to (17), wherein arginine or a physiologically acceptable salt thereof is administered at a concentration of 0.1 to 12 g per day.

(21) The method according to (17), wherein arginine or a physiologically acceptable salt thereof is administered at a concentration of 0.5 to 6 g per day.

(22) The method according to (17), wherein said disease caused by a decrease in erythropoiesis is selected from the group consisting of renal anemia, iron-deficiency anemia, hemolytic anemia, aplastic anemia, pernicious anemia, bleeding anemia, and anemia accompanying a treatment with an anti-cancer agent.

(23) The method according to (17), wherein the hematopoietic factor is at least one selected from the group consisting of activin, SCF and IGF-1.

(24) The method according to (17), wherein said hematopoietic factor production promoter promotes production of hematopoietic factor in the bone marrow.

(25) A method of screening for an inhibitor for a hematopoietic factor production promoter which comprises arginine or a physiologically acceptable salt thereof as an active ingredient, wherein said method comprises:

(a-1) measuring hematopoietic factor production-promoting activities by adding the hematopoietic factor production promoter and a test substance to a hematopoietic factor production-promoting activity measurement system which is for measuring the hematopoietic factor production-promoting activity;

(a-2) measuring hematopoietic factor production-promoting activities by adding the hematopoietic factor production promoter to a hematopoietic factor production-promoting activity measurement system which is for measuring the hematopoietic factor production-promoting activity;

(b) comparing hematopoietic factor production-promoting activities of (a-1) to hematopoietic factor production-promoting activities of (a-2); and

(c) classifying the test substance as an inhibitor for a hematopoietic factor production promoter when the hematopoietic factor production-promoting activity of (a-1) is less than the hematopoietic factor production-promoting activity of (a-2).

(26) The method according to (25), wherein the hematopoietic factor production promoter and the test substance are added simultaneously.

(27) The method according to (25), wherein the hematopoietic factor production promoter and the test substance are added sequentially.

(28) The method according to (25), wherein said hematopoietic factor production-promoting activity measurement system is quantitative PCR.

(29) A method of reducing the production of hematopoietic factor in the bone marrow, comprising administering to a subject in need thereof an effective amount of a composition comprising the hematopoietic factor production inhibitor identified by the method according to (25).

(30) The method according to (29), wherein said administering is orally.

(31) The method according to (29), wherein said administering is parenterally.

(32) The method according to (29), wherein the hematopoietic factor is at least one selected from the group consisting of activin, SCF and IGF-1.

(33) A method of treating a disease caused by an increase in erythropoiesis, comprising administering to a subject in need thereof an effective amount of a composition comprising the hematopoietic factor production inhibitor identified by the method according to (25).

(34) The method according to (33), wherein said administering is orally.

(35) The method according to (33), wherein said administering is parenterally.

(36) The method according to (33), wherein the hematopoietic factor is at least one selected from the group consisting of activin, SCF and IGF-1.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1A is a graph showing the activin production-promoting action of arginine in bone marrow cells isolated from a renal failure rat.

FIG. 1B is a graph showing the SCF production-promoting action of arginine in bone marrow cells isolated from a renal failure rat.

FIG. 1C is a graph showing the IGF-1 production-promoting action of arginine in bone marrow cells isolated from a renal failure rat.

FIG. 2 is a graph showing the inhibition of CFU-E-colony stimulating activity of arginine by inhibitors for hematopoietic factors.

### DETAILED DESCRIPTION OF THE PREFERRED EMBODIMENTS

Unless specifically defined, all technical and scientific terms used herein have the same meaning as commonly understood by a skilled artisan in enzymology, biochemistry, cellular biology, molecular biology, and the medical sciences.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will control. Further, the materials, methods, and examples are illustrative only and are not intended to be limiting, unless otherwise specified.

The hematopoietic factor production promoter according to the present invention comprises arginine as an active ingredient. The arginine may be arginine and/or a physiologically acceptable salt thereof. Examples of suitable salts include acid addition salts, such as a hydrochloride, a hydrobromide, a hydroiodide; acid addition salts of arginine with citric acid, sulfuric acid, phosphoric acid, methanesulfonic acid, benzenesulfonic acid, toluenesulfonic acid; acid addition salts of arginine with an acidic amino acid such as glutamic acid and aspartic acid; and the like. Other suitable salts include a sodium salt, a potassium salt, an ammonium salt, a mono-, di-, or trialkylammonium salt, a mono-, di-, or tri(hydroxyalkyl)ammonium salt, etc. However, the salt is not limited to the foregoing examples. As for an isomer of the active ingredient, any of the L-form, D-form and DL-form can be used, however, the L-form is preferred because it is naturally occurring. For example, as the arginine, the free-form of L-arginine and L-arginine monohydrochloride may be included in combination.

The hematopoietic factor production promoter of the present invention can be prepared in any of known forms as well as various forms of pharmaceutical preparations to be discovered in the future for, for example, oral administration, intraperitoneal administration, percutaneous administration, subcutaneous administration, intravenous administration, inhalation, and the like. In the myeloerythroid progenitor differentiation inducer of the present invention, arginine can be used alone. However, as a pharmaceutical preparation comprising arginine as an active ingredient, it can be prepared in any of various forms by suitably employing known methods and methods developed in the future.

A method of administering the hematopoietic factor production promoter of the present invention is not particularly limited, however, oral administration is preferred. In this case, the dose varies depending on the hemoglobin level, which becomes an indicator of anemia for a patient to be administered, the age and the like, however, in the case of adults, it is about 0.1 to 12 g, more preferably about 0.5 to 6 g per day.

The hematopoietic factor production promoter of the present invention can be used as an active ingredient of a pharmaceutical product to be used for treating or preventing a variety of diseases caused by decrease in erythropoiesis or a constituent of a food or a medical food. Examples of the disease in which the hematopoietic factor production promoter of the present invention is expected to be effective include renal anemia, iron-deficiency anemia, hemolytic anemia, aplastic anemia, pernicious anemia, bleeding anemia and anemia accompanying a treatment with an anti-cancer agent.

As a method of applying the active ingredient of the present invention to a pharmaceutical, oral administration or parenteral administration can be employed. However, upon administration, the active ingredient is mixed with a solid or liquid nontoxic carrier for pharmaceutical use which is suitable for the administration route such as oral administration or injection, whereby it can be administered in a common dosage form for a pharmaceutical preparation. Examples of such a pharmaceutical preparation include solid preparations such as tablets, granules, powders and capsules, liquid preparations such as solutions, suspensions and emulsions, lyophilized preparations, and the like. These pharmaceutical preparations can be prepared in a customary manner. Examples of the nontoxic carrier for pharmaceutical use include glucose, lactose, sucrose, starches, mannitol, dextrins, glycerides of fatty acids, polyethylene glycol, hydroxyethyl starches, ethylene glycol, polyoxyethylene sorbitan fatty acid esters, amino acids, gelatin, albumin, water, physiological saline, and the like. Further, a commonly used additive such as a stabilizer, a lubricant, an emulsifying agent, a binder, or a tonicity adjusting agent can be used as needed.

By using the hematopoietic factor production promoter of the present invention, an agonist for the promoter (i.e., a substance that enhances the hematopoietic factor production-promoting activity that the hematopoietic factor production promoter of the present invention, hereinafter also referred to as a “hematopoietic factor production-promoting activity enhancer”) can be screened. It can be expected that such a hematopoietic factor production-promoting activity enhancer is developed as a therapeutic agent for a disease that is caused by the lack of promotion of hematopoiesis due to the promotion of hematopoietic factor production by arginine present in vivo. Herein, the phrase “hematopoietic factor production-promoting activity” refers to an activity of elevating production of a certain molecule via the gene expression of a hematopoietic factor.

The hematopoietic factor production-promoting activity enhancer can be screened by, for example, the following steps. However, the method is not limited to these steps. The steps include:

i) measuring hematopoietic factor production- promoting activities by adding arginine or arginine and a test substance to a hematopoietic factor production-promoting activity measurement system which is for measuring the hematopoietic factor production-promoting activity;

ii) comparing the hematopoietic factor production-promoting activity in the case of adding only arginine to the measurement system with the hematopoietic factor production-promoting activity in the case of adding arginine and a test substance to the measurement system; and

iii) selecting the test substance exhibiting enhanced (i.e., increased) hematopoietic factor production-promoting activity when the test substance is added.

By way of Example, in the measuring step (i) above, the following are envisioned:

(a-1) measuring hematopoietic factor production-promoting activities by adding the hematopoietic factor production promoter and a test substance to a hematopoietic factor production-promoting activity measurement system which is for measuring the hematopoietic factor production-promoting activity; and

(a-2) measuring hematopoietic factor production-promoting activities by adding the hematopoietic factor production promoter to a hematopoietic factor production-promoting activity measurement system which is for measuring the hematopoietic factor production-promoting activity.

By way of Example, in the measuring step (ii) above, the following is envisioned comparing hematopoietic factor production-promoting activities of (a-1) to hematopoietic factor production-promoting activities of (a-2).

By way of Example, in the measuring step (iii) above, the following is envisioned classifying the test substance as an enhancer for a hematopoietic factor production promoter when the hematopoietic factor production-promoting activity of (a-1) is greater than the hematopoietic factor production-promoting activity of (a-2).

By using the hematopoietic factor production promoter of the present invention, an antagonist for the promoter (i.e., a substance that inhibits the hematopoietic factor production-promoting activity that the hematopoietic factor production promoter of the present invention, hereinafter also referred to as a “hematopoietic factor production-promoting activity enhancer”) can be screened. It can be expected that such a hematopoietic factor production-promoting activity inhibitor is developed as a therapeutic agent for a disease wherein the promotion of hematopoiesis due to the promotion of hematopoietic factor production by arginine present in vivo is associated with the disease.

The hematopoietic factor production-promoting activity inhibitor can be screened by, for example, the following steps. However, the method is not limited to these steps. The steps include:

i) measuring hematopoietic factor production-promoting activities by adding arginine or arginine and a test substance to a hematopoietic factor production-promoting activity measurement system which is for measuring the hematopoietic factor production-promoting activity;

ii) comparing the hematopoietic factor production-promoting activity in the case of adding only arginine to the measurement system with the hematopoietic factor production-promoting activity in the case of adding arginine and a test substance to the measurement system; and

iii) selecting the test substance exhibiting an inhibited (i.e., decreased) hematopoietic factor production-promoting activity when the test substance is added.

By way of Example, in the measuring step (i) above, the following are envisioned:

(a-1) measuring hematopoietic factor production-promoting activities by adding the hematopoietic factor production promoter and a test substance to a hematopoietic factor production-promoting activity measurement system which is for measuring the hematopoietic factor production-promoting activity; and

(a-2) measuring hematopoietic factor production-promoting activities by adding the hematopoietic factor production promoter to a hematopoietic factor production-promoting activity measurement system which is for measuring the hematopoietic factor production-promoting activity.

By way of Example, in the measuring step (ii) above, the following is envisioned comparing hematopoietic factor production-promoting activities of (a-1) to hematopoietic factor production-promoting activities of (a-2).

By way of Example, in the measuring step (iii) above, the following is envisioned classifying the test substance as an inhibitor for a hematopoietic factor production promoter when the hematopoietic factor production-promoting activity of (a-1) is less than the hematopoietic factor production-promoting activity of (a-2).

The measurement of the hematopoietic factor production-promoting activity is performed by using, for example, cells isolated from an animal. The cells and the hematopoietic factor production-promoting activity measurement system can be used without being particularly limited as long as they enable the detection of gene expression and protein production after the addition of a test substance.

As the “hematopoietic factor production-promoting activity measurement system,” in addition to the quantitative PCR method described in Examples and the like, a gene chip method, a microarray method, in situ hybridization, RNase protection assay, Northern blotting, the differential display method, SDS acrylamide gel electrophoresis, Western blotting, column chromatography and the like can be employed.

The hematopoietic factor production promoter according to the present invention may comprise either arginine or a physiologically acceptable salt thereof, or may comprise an arbitrary mixture thereof. Further, the concentration of the test substance may be arbitrary as long as it does not affect the hematopoietic factor production-promoting activity measurement system. In addition, the test substance may be a single compound or a mixture or a composition containing a plurality of compounds.

Incidentally, arginine and the test substance are generally added to the hematopoietic factor production-promoting activity measurement system simultaneously, however, as long as the hematopoietic factor production-promoting activity of arginine can be detected, they may not be added simultaneously. In other words, the present invention also encompasses sequential addition of the arginine and the test substance, in either order thereof.

By the above-mentioned procedure, or by repeating the above-mentioned procedure, a substance inhibiting or enhancing the hematopoietic factor production-promoting activity that the hematopoietic factor production promoter of the present invention has, a composition containing such a substance or the like can be screened and identified.

The hematopoietic factor production-promoting activity inhibitor and the hematopoietic factor production-promoting activity enhancer selected as described above are expected as a new substance for regulating the hematopoietic factor production-promoting activity of arginine, a therapeutic agent for a variety of diseases associated with the hematopoietic factor production-promoting activity or a candidate thereof.

Accordingly, the present invention provides a hematopoietic factor production promoter which has high safety, can be orally administered and is widely used.

It is expected that the hematopoietic factor production promoter of the present invention enables prevention or treatment of anemia for patients with anemia, and clinical symptoms such as less incentive to work, fatigability, shortness of breath, lightheadedness and palpitation, each of which accompanies anemia, are improved.

Further, if the hematopoietic factor production promoter of the present invention can be orally administered, the pain due to subcutaneous or intravenous injection is not caused, and moreover, it can be easily taken daily at home without going to the hospital. Therefore, it is expected that the promoter can achieve reliable improvement of anemia. Further, arginine is an amino acid present in vivo, therefore it is expected that side effects accompanying its intake do not occur and treatment can be performed without concerning the worsening of the renal function.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials.

Where a numerical limit or range is stated herein, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

Having generally described this invention, a further understanding can be obtained by reference to certain specific examples, which are provided herein for purposes of illustration only, and are not intended to be limiting unless otherwise specified.

### EXAMPLES

### Example 1

### Activin, SCF and IGF-1 Production-promoting Activity of Arginine in Bone Marrow Cells Isolated from Renal Failure Rat

A renal failure rat (a rat from which ⅚ of kidney was removed) was produced as follows. A male Wistar rat at 8 weeks of age (Charles River Japan, Inc.) was subjected to the total extirpation of the right kidney and the ⅔ extirpation of the renal cortex of the left kidney under anesthesia with Nembutal and was sutured. After the surgery, the rat was housed under normal housing condition (CRF-1 diet, Charles River Japan, Inc.). Six weeks after the surgery the renal failure rat was killed by diethyl ether, then both femurs were excised and bone marrow cells were isolated. The isolated bone marrow cells were suspended in an IMDM medium with blood amino acid concentration containing 10% FCS. As for the IMDM medium with blood amino acid concentration, an IMDM medium in which the amino acid composition was changed to a composition shown in Table 1 was prepared and used.

Ten milliliters of the bone marrow cell suspension prepared at 2×10⁶ cells/ml was cultured in a dish with a diameter of 10 cm (Nalge Nunc, Inc.) under the condition of 37° C. and 5% CO₂ for 24 hours. Twenty-four hours after the initiation of culture, arginine was added thereto. After 2 hours, the bone marrow cells in the dish were colleted and used for preparation of total RNA and cDNA synthesis.

TABLE 1

| No. | Amino acid | Concentration (μM) |
|---|---|---|
| 1 | L-Arginine | 100 |
| 2 | L-Asparagine | 50 |
| 3 | L-Aspartic acid | 10 |
| 4 | L-Cystine | 25 |
| 5 | L-Glutamic acid | 60 |
| 6 | L-Glutamine | 600 |
| 7 | Glycine | 250 |
| 8 | L-Histidine | 70 |
| 9 | Alanine | 387 |
| 10 | L-Isoleucine | 75 |
| 11 | L-Leucine | 130 |
| 12 | L-Lysine | 260 |
| 13 | L-Methionine | 45 |
| 14 | L-Phenylalanine | 60 |
| 15 | L-Proline | 130 |
| 16 | L-Serine | 140 |
| 17 | L-Threonine | 160 |
| 18 | L-Tryptophan | 50 |
| 19 | L-Tyrosine | 70 |
| 20 | L-Valine | 200 |

Total RNA preparation and cDNA synthesis were performed by the following methods. Bone marrow cells corresponding to one femur were suspended in 1.5 ml of Isogen (Nippon Gene Co., Ltd.) and homogenized (BIO 101 FastPrep). Total RNA was purified using 1 ml of the homogenate in accordance with the method described in Isogen. The quality of the total RNA was confirmed by Bioanalyzer (Japan Becton Dickinson). By using 5 μg of the total RNA as a template, cDNA synthesis was carried out using an oligo dT 20mer primer (Invitrogen) and SuperScript III (Invitrogen). The method of cDNA synthesis was in accordance with the method described in SuperScript III.

The quantification of mRNA of each of the hematopoietic factors was determined by the quantitative PCR method mentioned below. Based on the sequences of activin (rat Inhibin betaA), SCF and IGF-1 registered in NCBI (GenBank accession Nos. M37482, NM021843, and NM178886, respectively in the order), oligonucleotide primers to be used for the quantitative PCR method were synthesized. The primers described in SEQ ID NOS: 1 and 2 in Table 2, the primers described in SEQ ID NOS: 3 and 4 in Table 2, and the primers described in SEQ ID NOS: 5 and 6 in Table 2 were used for activin gene, SCF gene, and IGF-1 gene, respectively.

The quantitative PCR method was in accordance with the method described in SYBRGreen PCR kit (TOYOBO., Ltd.). More specifically, a PCR reaction (after 1 minute at 94° C., 40 cycles of 30 seconds at 94° C. and 1 minute at 60° C.) with an ABI7700 (Applied Biosystems Japan Ltd.) using 1 μl of a cDNA solution as a template, 500 nM of the primers, and a SYBRGreen PCR kit and the detection of amplified product were performed and the relative concentration of mRNA was calculated. After the quantitative PCR, the presence or absence and size of an amplified product were confirmed by agarose gel electrophoresis.

TABLE 2

| | |
|---|---|
| SEQ ID NO | Oligonucleotide primer |

| | |
|---|---|
| 1 | gaaaacgggttatgtggaga |
| 2 | tgaaacagacggatggtga |
| 3 | tggtggacgctcttcagtt |
| 4 | catctccagcctcctcaga |
| 5 | ctacaatggacagcaat |
| 6 | gaaactctctctctttctgttgc |

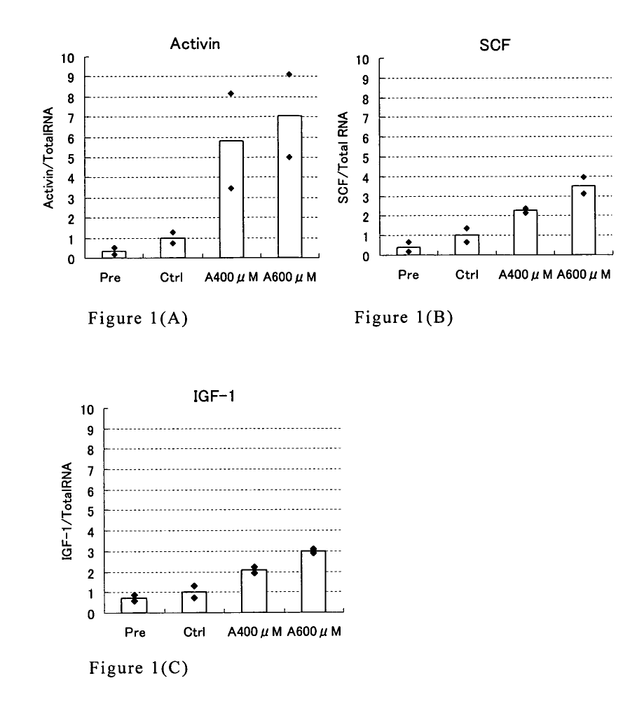

Bone marrow cells were isolated from a renal failure rat, and the bone marrow cells were collected 2 hours after the addition of arginine, and then mRNA of each of activin, SCF and IGF-1 was quantified by the quantitative PCR method. The results are shown in FIGS. 1A to 1C. The data is expressed as a mean value ±SEM. The vertical axis indicates a relative mRNA level per total RNA.

Pre: Before the addition of arginine, arginine concentration: 100 μM

Ctrl: 2 hours without the addition of arginine, arginine concentration: 100 μM

A400 μM: 2 hours with the addition of arginine, arginine concentration: 400 μM

A600 μM: 2 hours with the addition of arginine, arginine concentration: 600 μM

From these results, it was determined that arginine directly acts on a bone marrow cell and promotes the production of activin, SCF and IGF-1.

### Example 2

### Inhibition of CFU-E-colony Stimulating Activity of Arginine by Activin, SCF, or IGF-1 Inhibitor

An in vitro mouse CFU-E colony assay was conducted in accordance with the following method. After a female BDF-1 mouse at 10 weeks of age (Charles River Japan, Inc.) was killed by the cervical dislocation method, bone marrow cells were isolated from the femur and suspended in an IMDM medium (Invitrogen) containing 10% FCS (JRH Bioscience Inc). The bone marrow cells were centrifuged at 1500 rpm for 10 minutes at 4° C. and the precipitated bone marrow cells were resuspended in an amino acid-free IMDM medium and the number of cells was measured.

To a dish with a diameter of 3.5 cm (Nalge Nunc, Inc. International), 1 ml of a methyl cellulose semi-solid medium in which the bone marrow cells were suspended {1 IU/ml rHuEPO (Chugai Pharmaceutical Co., Ltd.), 100 μM 2-mercaptoethanol (Wako Pure Chemical Industries, Ltd.), 24% FCS (JRH Bioscience Inc.), 0.8% methyl cellulose (methyl cellulose containing IMDM solution M3134, Stem Cell Technologies, Inc.), 2% BSA (SIGMA-ALDRICH Japan K.K.), 2.2×10⁵ cells/ml of bone marrow cells} containing arginine at a concentration of 170 μM was added (which is equal to an IMDM medium whose concentration of the amino acid composition is one-third and which includes FCS at 24%). Under the presence of 170 μM arginine, arginine was further added to make a final concentration of 600 μM and follistatin (FSN, anti-activin, R&D Co., 100 ng/ml), anti-SCF antibody (Pepro Tech EC Ltd., 1 μg/ml) or anti-IGF-1 antibody (Pepro Tech EC Ltd., 2 μg/ml) was further added. At 28 hours after the culture was carried out under the condition of 37° C. and 5% CO₂, the number of CFU-E colonies was measured using an inverted microscope. N=4 for each condition. Unpaired t-test was used for a statistical analysis. * indicates P<0.05, and ** indicates P<0.005. The data is expressed as a mean value±SEM.

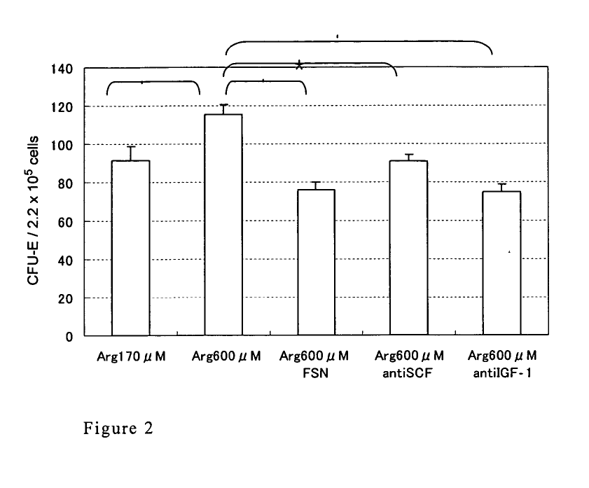

The results of CFU-E colony assay are shown in FIG. 2. The vertical axis indicates the number of CFU-E colonies per 2.2×10⁵ bone marrow cells.

From FIG. 2, it was determined that the CFU-E-colony stimulating activity of arginine is inhibited by follistatin, anti-SCF antibody and anti-IGF-1 antibody. Accordingly, it was revealed that arginine acts on a bone marrow cell and increases CFU-E via the promotion of production of activin, SCF and IGF-1, which are hematopoietic factors.

Numerous modifications and variations on the present invention are possible in light of the above teachings. It is, therefore, to be understood that within the scope of the accompanying claims, the invention may be practiced otherwise than as specifically described herein.

1. A method of screening for an enhancer for a hematopoietic factor production promoter which comprises arginine or a physiologically acceptable salt thereof as an active ingredient, wherein said method comprises:
(a-1) measuring hematopoietic factor production-promoting activities by adding the hematopoietic factor production promoter and a test substance to a hematopoietic factor production-promoting activity measurement system which is for measuring the hematopoietic factor production-promoting activity;
(a-2) measuring hematopoietic factor production-promoting activities by adding the hematopoietic factor production promoter to a hematopoietic factor production-promoting activity measurement system which is for measuring the hematopoietic factor production-promoting activity;
(b) comparing hematopoietic factor production-promoting activities of (a-1) to hematopoietic factor production-promoting activities of (a-2); and
(c) classifying the test substance as an enhancer for a hematopoietic factor production promoter when the hematopoietic factor production-promoting activity of (a-1) is greater than the hematopoietic factor production-promoting activity of (a-2).

2. The method according to claim 1, wherein the hematopoietic factor production promoter and the test substance are added simultaneously.

3. The method according to claim 1, wherein the hematopoietic factor production promoter and the test substance are added sequentially.

4. The method according to claim 1, wherein said hematopoietic factor production-promoting activity measurement system is quantitative PCR.

5. A method of enhancing production of hematopoietic factor in the bone marrow, comprising administering to a subject in need thereof an effective amount of a composition comprising a hematopoietic factor production promoter comprising arginine or a physiologically acceptable salt thereof and an enhancer for the hematopoietic factor production promoter identified by the method according to claim 1.

6. The method according to claim 5, wherein said administering is orally.

7. The method according to claim 5, wherein said administering is parenterally.

8. The method according to claim 5, wherein arginine or a physiologically acceptable salt thereof is administered at a concentration of 0.1 to 12 g per day.

9. The method according to claim 5, wherein arginine or a physiologically acceptable salt thereof is administered at a concentration of 0.5 to 6 g per day.

10. The method according to claim 5, wherein the hematopoietic factor is at least one selected from the group consisting of activin, SCF and IGF-1.

11. A method of treating a disease caused by a decrease in erythropoiesis, comprising administering to a subject in need thereof an effective amount of a composition comprising a hematopoietic factor production promoter comprising arginine or a physiologically acceptable salt thereof and an enhancer for the hematopoietic factor production promoter identified by the method according to claim 1.

12. The method according to claim 11, wherein said administering is orally.

13. The method according to claim 11, wherein said administering is parenterally.

14. The method according to claim 11, wherein arginine or a physiologically acceptable salt thereof is administered at a concentration of 0.1 to 12 g per day.

15. The method according to claim 11, wherein arginine or a physiologically acceptable salt thereof is administered at a concentration of 0.5 to 6 g per day.

16. The method according to claim 11, wherein said disease caused by a decrease in erythropoiesis is selected from the group consisting of renal anemia, iron-deficiency anemia, hemolytic anemia, aplastic anemia, pernicious anemia, bleeding anemia, and anemia accompanying a treatment with an anti-cancer agent.

17. The method according to claim 11, wherein the hematopoietic factor is at least one selected from the group consisting of activin, SCF and IGF-1.

18. The method according to claim 11, wherein said hematopoietic factor production promoter promotes production of hematopoietic factor in the bone marrow.

19. A method of screening for an inhibitor for a hematopoietic factor production promoter which comprises arginine or a physiologically acceptable salt thereof as an active ingredient, wherein said method comprises:
(a-1) measuring hematopoietic factor production-promoting activities by adding the hematopoietic factor production promoter and a test substance to a hematopoietic factor production-promoting activity measurement system which is for measuring the hematopoietic factor production-promoting activity;
(a-2) measuring hematopoietic factor production-promoting activities by adding the hematopoietic factor production promoter to a hematopoietic factor production-promoting activity measurement system which is for measuring the hematopoietic factor production-promoting activity;
(b) comparing hematopoietic factor production-promoting activities of (a-1) to hematopoietic factor production-promoting activities of (a-2); and
(c) classifying the test substance as an inhibitor for a hematopoietic factor production promoter when the hematopoietic factor production-promoting activity of (a-1) is less than the hematopoietic factor production-promoting activity of (a-2).

20. The method according to claim 19, wherein the hematopoietic factor production promoter and the test substance are added simultaneously.

21. The method according to claim 19, wherein the hematopoietic factor production promoter and the test substance are added sequentially.

22. The method according to claim 19, wherein said hematopoietic factor production-promoting activity measurement system is quantitative PCR.

23. A method of reducing the production of hematopoietic factor in the bone marrow, comprising administering to a subject in need thereof an effective amount of a composition comprising the hematopoietic factor production inhibitor identified by the method according to claim 19.

24. The method according to claim 23, wherein said administering is orally.

25. The method according to claim 23, wherein said administering is parenterally.

26. The method according to claim 23, wherein the hematopoietic factor is at least one selected from the group consisting of activin, SCF and IGF-1.

27. A method of treating a disease caused by an increase in erythropoiesis, comprising administering to a subject in need thereof an effective amount of a composition comprising the hematopoietic factor production inhibitor identified by the method according to claim 19.

28. The method according to claim 27, wherein said administering is orally.

29. The method according to claim 27, wherein said administering is parenterally.

30. The method according to claim 27, wherein the hematopoietic factor is at least one selected from the group consisting of activin, SCF and IGF-1.
